(19) United States
(12) Patent Application Publication
(10) Pub. No.: US 2023/0276838 A1
(43) Pub. Date: Sep. 7, 2023

(54) Process for Applying Additives To Aerosol Generating Substrates and Products Made Therefrom

(71) Applicant: Mativ Holdings, Inc., Alpharetta, GA (US)

(72) Inventors: Sandra de Jongh, Le Mans (FR); Laurent Gombert, Le Mans (FR)

(21) Appl. No.: 18/116,127

(22) Filed: Mar. 1, 2023

Related U.S. Application Data
(60) Provisional application No. 63/315,699, filed on Mar. 2, 2022.

(51) Int. Cl. A24B 15/30; A24B 15/167; A24B 15/38; A24B 15/40; A24F 40/20; A61K 9/00; A61K 31/05; A61K 31/352

(52) U.S. Cl. CPC A24B 15/303; A24B 15/167; A24B 15/38; A24B 15/406; A24F 40/20; A61K 9/0073; A61K 31/05; A61K 31/352

(57) ABSTRACT

A process is disclosed for incorporating additives into aerosol generating products. During the process, an additive is incorporated into an aerosol modifying composition. An aerosol generating filler is contacted with the aerosol modifying composition and the additive is uniformly incorporated into the substrate. In one application, a cannabidiol is incorporated into a tobacco filler or a cannabaceae filler.

### RELATED APPLICATIONS

The present application is based upon and claims priority to U.S. Provisional Patent Application Ser. No. 63/315,699, having a filing date of Mar. 2, 2022, and which is incorporated herein by reference.

### BACKGROUND

Conventional smoking articles combust a material at temperatures that release active compounds, which are inhaled through the mainstream smoke. The mainstream smoke delivered to the user not only has a characteristic, enjoyable, taste, but also can deliver to the user active compounds that are absorbed into the blood through the lungs that can provide the smoker with a pleasant and calming effect. In addition to smoking articles that are consumed with a lit end, other aerosol-producing articles also exist in which an aerosol, such as a vapor, is produced without igniting the material. For example, heat but not burn sticks are consumed similar to conventional smoking articles without igniting a filler contained in the stick.

In the past, problems have been experienced in delivering active compounds from a smoking article or heat but not burn stick to a user in controlled amounts. For example, it has been difficult to control the amount of active compounds delivered to a user by an article, and harder yet to maintain good sensory characteristics, such as taste and smell, while modifying the amount of active compounds contained in the article.

Additional problems have been encountered when attempting to incorporate active compounds derived from cannabaceae materials into smoking articles or heat but not burn sticks. For example, metering levels of tetrahydrocannabinol (THC) and/or cannabidiol (CBD) while maintaining a good or authentic taste has been difficult to achieve. For instance, THC and/or CBD deliveries can vary dramatically depending upon the particular plant and the particular plant parts being incorporated, further increasing the difficulty in controlling delivery.

In view of the above, a need currently exists for a method of incorporating an additive into an aerosol generating material. More particularly, a need exists for a method that can not only efficiently incorporate an additive into an aerosol generating material but also in a manner such that the additive is uniformly dispersed throughout the material, if desired. In one aspect, a need also exists for a method of incorporating an additive into an aerosol generating material at relatively high levels that were difficult to obtain using past processes.

### SUMMARY

In general, the present disclosure is directed to an aerosol generating product comprising a plant containing substrate. The plant containing substrate may be impregnated with an aerosol modifying composition. In one aspect, the aerosol modifying composition may include an additive and surfactant mixture. For instance, the additive may comprise a cannabinoid, a terpene, an essential oil, or mixtures thereof. The plant containing substrate may comprise cannabaceae fibers, tobacco fibers, botanical fibers, bast pulp fibers, wood pulp fibers, or mixtures thereof. The surfactant may comprise a non-ionic surfactant. For instance, the surfactant may comprise a polysorbate, an ethoxylated oil, a biosurfactant, or mixtures. The additive, for instance, may be incorporated into the plant containing substrate in an amount greater than about 1% by weight.

In one embodiment, the plant containing substrate may comprise a reconstituted material. In another embodiment, the plant containing substrate may comprise cannabaceae fibers, tobacco fibers, botanical fibers, bast pulp fibers, wood pulp fibers, or mixtures thereof.

In an additional embodiment, the surfactant may comprise an ethoxylated sorbitan of a fatty acid. In another embodiment, the surfactant may comprise a polyoxyethylene sorbitan monolaurate, a polyoxyethylene sorbitan monopalmitate, a polyoxyethylene sorbitan monostearate, a polyoxyethylene sorbitan monostearate, a polyoxyethylene sorbitan monooleate, or mixtures thereof. In yet another embodiment, the surfactant may comprise an ethoxylate of a hydrogenated castor oil.

In one embodiment, the additive may comprise a cannabinoid, which includes phytocannabinoids and synthetic cannabinoids. Examples of cannabinoids include all types of cannabidiol (CBD) and all types of tetrahydrocannabinol (THC). Other cannabinoids may include cannabigerol type (CBG), cannabichromene type (CBC), cannabicyclol type (CBL), cannabielsoin type (CBE), cannabinol type (CBN), cannabinodiol type (CBND), cannabitriol type (CBT), cannabifuran (CBF), CBG cannabigerol, delta-10-tetrahydrocannabinol, CBGA cannabigerolic acid, CBDA cannabidiolic acid, CBCA cannabichromenic acid, and all types of THCAs tetrahydrocannabinolic acids.

In one aspect, the additive may comprise an essential oil and/or plant extract.

Furthermore, in an embodiment, the additive may comprise a terpene. The terpene can comprise alpha-pinene, beta-pinene, camphene, delta-3-carene, beta-myrcene, alpha-terpinene, D-limonene, terpineol, borneol, gamma-terpinene, terpinolene, linalool, isopulegol, geraniol, beta-caryophyllene, alpha-humulene, caryophyllene oxide, nerolidol, guaiol, alpha-bisabolol or mixtures thereof.

Additionally or alternatively, the additive is present in the plant containing substrate in an amount greater than about 2% by weight, such as greater than about 3% by weight, such as greater than about 5% by weight, such as greater than about 8% by weight, such as greater than about 10% by weight and less than about 50% by weight, such as less than about 40% by weight, such as less than about 30% by weight, such as less than about 20% by weight, such as less than about 15% by weight.

In another embodiment, the dried plant containing substrate has a basis weight of from about 10 gsm to about 200 gsm, such as from about 20 gsm to about 150 gsm. In yet another embodiment, the plant containing substrate may include a filler material comprising a strip, strips, shreds, or mixtures thereof of a reconstituted material.

The present disclosure is also directed to a process for producing a component for an aerosol generating product. The component may comprise a wrapper or an aerosol producing filler. The process comprises applying to a plant containing substrate an aerosol modifying composition and drying the plant containing substrate. The aerosol modifying composition comprising the additive and a surfactant mixture may be blended with water to form an aqueous-based dispersion. The additive is incorporated into the plant containing substrate in an amount greater than about 1% by weight.

In one embodiment, the additive and surfactant mixture may be heated to a temperature of about 40° C. to about 90° C. In yet another embodiment, the additive and surfactant mixture contain the additive in an amount of from about 1 percent by weight to about 90 percent by weight, such as from about 40 percent by weight to about 70 percent by weight, such as from about 55 percent by weight to about 65 percent by weight, based on the total weight of additive and surfactant.

The plant containing substrate, for instance, is formed in a wetlaid process and the aerosol modifying composition is applied to the plant containing substrate during formation of the substrate. Additionally or alternatively, the plant containing substrate is formed in a wet laid process and the aerosol modifying composition is applied to the plant containing substrate using a size press. For example, the plant containing substrate can be submerged in a bath containing the aerosol modifying composition and then fed through press rollers to apply the aerosol modifying composition or is fed through a flooded nip in order to apply the aerosol modifying composition.

In a further embodiment, the aerosol modifying composition may comprise an extract obtained from the plant containing substrate.

In yet another embodiment, the plant containing substrate may comprise a reconstituted cannabaceae material. For instance, the reconstituted cannabaceae material may include extracted cannabaceae fibers comprising cannabaceae leaves, cannabaceae hurds, cannabaceae buds, cannabaceae flowers, or mixtures thereof. Additionally, the reconstituted cannabaceae material may be combined with wood pulp fibers, bast pulp fibers, or mixtures thereof. In another embodiment, the plant containing substrate may comprise a reconstituted tobacco material or a reconstituted cocoa material.

Furthermore, in an embodiment, the plant containing substrate may be treated with a humectant. In an additional embodiment, the humectant may comprise glycerol, propylene glycol or mixtures thereof. The humectant is present, for instance, in the reconstituted cannabaceae material in an amount of about 5% by weight to about 50% by weight.

In yet another embodiment, the additive is present in the dried plant containing substrate in an amount greater than about 2% by weight, such as greater than about 5% by weight, such as greater than about 10% by weight, such as greater than about 15% by weight, such as greater than about 20% by weight and less than about 30% by weight.

In a further embodiment, the plant containing substrate is tobacco-free.

Additionally or alternatively, in one embodiment, the plant containing substrate comprises a wrapper. The wrapper can comprise a substrate having a basis weight of from about 14 gsm to about 80 gsm and having a permeability of from about 10 CORESTA to about 100 CORESTA. In another embodiment, the fibrous web contains filler particles in an amount from about 1% to about 40% by weight, such as in an amount from about 5% to about 20% by weight. In a further embodiment, the plant containing substrate comprises a cast leaf product.

In general, the present disclosure is also generally directed to a smoking article comprising an outer wrapper surrounding a smokable rod, the outer wrapper or the smokable rod comprising the plant containing substrate of the present disclosure.

Generally speaking, the present disclosure is also directed to an aerosol generating device comprising a heating device and a chamber, the chamber containing the plant containing substrate of the present disclosure. In one embodiment, the heating device is positioned so as to heat the plant containing substrate for producing an inhalable aerosol containing the additive without burning the plant containing substrate.

Other features and aspects of the present disclosure are discussed in greater detail below.

### DEFINITIONS

As used herein, a “reconstituted plant material” refers to a material formed by a process in which a plant feed stock, such as Cannabis components, is extracted with a solvent to form an extract of solubles, such as water solubles, and an extracted insoluble portion or residue comprising fibrous material. The extracted and insoluble fibrous material is then formed into a sheet through any suitable process and the extract may either be discarded or reapplied to the formed sheet. The extract can be fed through various processes for concentrating the extract and optionally removing or adding various components prior to being recombined with the fibrous material. In the present disclosure, the reconstituted plant material can be formed from extracted plant fibers (e.g. Cannabis fibers, tobacco fibers, pulp fibers, botanical fibers or mixtures) optionally combined with web building fibers, such as cellulose fibers. The extract of solubles obtained from the plant fibers is optionally reapplied to the sheet or material.

As used herein, an “aerosol generating material” is meant to include both a combustible material that undergoes combustion in a smoking article and to an aerosol-forming material that is heated but not combusted to form an inhalable aerosol. Combustible smoking articles can include, without limitation, cigarettes, roll-your-own cigarettes, make-your-own cigarette, cigarillos, blunts, cigars, and the like. In a combustible product or a heat not burn stick, the aerosol generating material is surrounded by a wrapping material to form a smokable rod. Aerosol generating devices for generating an aerosol include, for instance, devices in which an aerosol is generated by electrical heating or by the transfer of heat from a combustible fuel element or heat source to heat but not burn the aerosol generating material or by a vaporized e liquid, which releases volatile compounds. As the released compounds cool, they condense to form an aerosol that is inhaled by the consumer.

As used herein, “extracted plant fibers” refers to plant fibers that have been subjected to an extraction process in which the plant has been contacted with an aqueous solution to remove water soluble components contained in the plant. The extraction process is different from a delignification process and from a bleaching treatment.

As used herein, “extracted byproducts” refer to Cannabis biomass that has been subjected to an extraction process for removing selected components, such as cannabinoids, without removing a substantial amount of water soluble components. The extracted byproducts can be referred to as biomass resulting from an extraction process where the extractant is a solvent, such as ethanol, a supercritical fluid such as carbon dioxide, a lipid such as a vegetable oil, or the like. Extracted byproducts, in accordance with the present disclosure, can be subjected to a second extraction process for removing water soluble components during the process of making a reconstituted plant material. Extracted byproducts well suited for use in the present disclosure include those that contain water soluble components in an amount greater than about 8% by weight, such as in an amount greater than about 12% by weight, such as in an amount greater than about 18% by weight, such as in an amount greater than about 24% by weight, such as in an amount greater than about 30% by weight, such as in an amount greater than about 40% by weight, such as in an amount greater than about 45% by weight and generally less than about 65% by weight, such as less than about 55% by weight.

As used herein, “delignified” cellulosic fibers (e.g., pulp fibers) refers to fibers that have been subjected to a pulping or delignification process by which the cellulose fibers are separated from the plant material through chemical means, mechanical means, or through a combination of chemical and mechanical means.

As used herein, the term “refine” is used to mean that the plant material is subjected to a mechanical treatment that modifies the fibers of the material so that they are better suited to forming a fibrous sheet or substrate. For example, refining can separate/individualize the fibers and fibrillate the fibers, which promotes hydrogen bonding between fibers and/or fibrills and increases the web strength. Refining can be accomplished using a conical refiner, disks refiner, or a beater such as a Valley beater. The mechanical process exerts an abrasive and bruising action on the plant material such that the plant material is deformed and declustered. Refining is a different process than delignification and pulping.

As used herein, the term “stalk” is used to refer to the main structural portion of a plant that remains after the leaves have been removed.

As used herein, the term “hurd” is used herein to refer to the structural portion of a plant, e.g., stem connecting the leaves or laminae to the stalk and also to the veins or ribs that extend through the leaves. The term “hurd” does not encompass the term “stalk” and vice versus.

As used herein, “cannabaceae” may refer to any variety of the Cannabis plant or hemp plant, such as Cannabis sativa, Cannabis sativa L, or Cannabis indica, for instance. More particularly, the present disclosure may refer to leaves, stems, seeds and flowers or any other part of the Cannabaceae plant.

### DETAILED DESCRIPTION

It is to be understood by one of ordinary skill in the art that the present discussion is a description of exemplary embodiments only and is not intended as limiting the broader aspects of the present disclosure.

The present disclosure is generally directed to an aerosol generating product comprising a plant containing substrate that has been treated with at least one additive. More particularly, the present disclosure is directed to an aerosol modifying composition that is applied to, such as being impregnated into, a plant containing substrate. The aerosol modifying composition contains at least one additive in combination with at least one surfactant to form a surfactant and additive mixture. The additive, for example, can comprise a cannabinoid, a terpene, an essential oil, mixtures thereof or the like. In one embodiment, the surfactant and additive mixture can be blended with water or an aqueous-based liquid that facilitates application of the additive to the plant containing substrate. For instance, the surfactant and additive mixture can be applied to the plant containing substrate while the substrate is being made, requiring no extra process steps. In addition, the surfactant and additive mixture can be made entirely from food-grade and safe components. Of particular advantage, the surfactant and additive mixture permits application of the additive to the substrate such that the additive is uniformly applied to the substrate. The surfactant and additive mixture also permits controlled application of the additive to the substrate. In one embodiment, relatively great amounts (e.g. greater than 10%, such as greater than 15%, such as greater than about 18% by weight of the final product) of the additive can be incorporated into the substrate. It should be understood, however, that the process of the present disclosure can be used to apply and control any suitable amount of the additive onto the substrate. In one aspect, the additive is incorporated into the substrate in an amount of from about 5% by weight to about 15% by weight.

The plant containing substrate can be made from any suitable material that may be incorporated into an aerosol generating product. The substrate treated in accordance with the present disclosure may comprise a wrapper for an aerosol generating product or an aerosol producing filler. The substrate can be formed from dried plant material, extracted plant fibers, or mixtures thereof. The substrate can be made from cannabaceae materials, tobacco materials, botanical materials, wood pulp fibers, bast fibers and mixtures thereof.

In one embodiment, the plant containing substrate may be at least partially or even wholly formed from a reconstituted material containing extracted plant fibers. The reconstituted plant material produces an aerosol when heated or burned that can deliver the additive to the user as part of an inhaled aerosol. Of particular advantage, the plant containing substrate of the present disclosure is well suited to acting as a carrier for the one or more additives. In one embodiment, for instance, the plant containing substrate can be treated with an aerosol modifying composition in a manner such that an aerosol generated by the substrate can deliver, for instance, THC and/or CBD to a user in controlled, consistent amounts.

### Substrate Materials

The aerosol generating product of the present disclosure includes a plant containing substrate that can be a filler that produces an aerosol when heated or burned or can comprise a wrapper that surrounds the filler. The substrate can be made from all different types of plant material and can be made in various different ways. In one embodiment, dried plant material is treated in accordance with the present disclosure. When in the form of a wrapper, the substrate can comprise a paper. In an alternative embodiment, the substrate is formed from extracted plant fibers. For example, the substrate can comprise a reconstituted material. The plant material contained in the substrate can be derived from cannabaceae fibers, tobacco fibers, botanical fibers, bast pulp fibers, wood pulp fibers, or mixtures thereof.

### Cannabaceae Fibers

Plant fibers can be obtained from plants derived from the Cannabaceae family including all plants that contain a cannabinoid. Particular examples include but are not limited to Cannabis indica or Cannabis sativa, including hemp. For instance, fibers can be obtained from a Cannabis sativa plant species, such as Cannabis sativa L, whether or not the plant species contains detectable levels of THC. Many species of cannabaceae, for instance, contain THC. There are, however, cannabaceae plant strains that are particularly low in THC and typically referred to as industrial hemp. Industrial hemp, for instance, can contain THC in an amount less than about 1% by weight, such as in an amount less than about 0.5% by weight, such as in an amount less than about 0.3% by weight, such as in an amount less than about 0.2% by weight, such as in an amount less than about 0.1% by weight. Cannabaceae components used to produce a drug for medicinal or recreational use, on the other hand, can contain anywhere from 3% to over 20% by weight THC. For instance, Cannabaceae fibers can be obtained from Cannabis indica or from Cannabis sativa or Cannabis sativa L.

The Cannabaceae fibers can be obtained from cannabaceae leaves, cannabaceae hurds, cannabaceae buds, cannabaceae flowers, cannabaceae seeds, any by-product of cannabaceae extraction, or mixtures thereof.

Although the plant containing substrate can be made exclusively from leaves and hurds or can be made exclusively from buds and flowers, in one embodiment, the reconstituted material is made from a mixture of leaves and hurds combined with buds and/or flowers. For example, in one embodiment, the weight ratio between the leaves and hurds and the buds and/or flowers is from about 1:8 to about 8:1, such as from about 1:5 to about 5:1, such as from about 1:4 to about 4:1, such as from about 2:1 to about 1:2. In one embodiment, the ratio can be about 1:1.

In one embodiment, the plant containing substrate may contain leaves and hurds in an amount greater than about 10% by weight, such as in an amount greater than about 20% by weight, such as in an amount greater than about 30% by weight, and generally in an amount less than about 99% by weight, such as in an amount less than about 90% by weight, such as in an amount less than about 70% by weight, such as in an amount less than about 60% by weight, such as in an amount less than about 50% by weight, such as in an amount less than about 40% by weight. Similarly, the substrate may contain buds and/or flowers in an amount greater than about 10% by weight, such as in an amount greater than about 20% by weight, such as in an amount greater than about 30% by weight, such as in an amount greater than about 40% by weight, such as in an amount greater than about 50% by weight, such as in an amount greater than about 60% by weight, and generally in an amount less than about 80% by weight, such as in an amount less than about 70% by weight, such as in an amount less than about 60% by weight, such as in an amount less than about 50% by weight.

The total amount of Cannabaceae fibers contained in the plant containing substrate can depend upon the particular application and the desired result. Cannabaceae fibers, such as extracted Cannabaceae fibers, for instance, can be present in the plant containing substrate in an amount anywhere from 1% by weight to about 99.9% by weight, such as from about 5% by weight to about 90% by weight. For example, the cannabaceae fibers can be present in the plant containing substrate in an amount greater than about 10% by weight, such as in an amount greater than about 20% by weight, such as in an amount greater than about 30% by weight, such as in an amount greater than about 40% by weight, such as in an amount greater than about 50% by weight, such as in an amount greater than about 60% by weight, such as in an amount greater than about 70% by weight, such as in an amount greater than about 80% by weight, and generally in an amount less than about 90% by weight, such as in an amount less than about 85% by weight, such as in an amount less than about 80% by weight.

### Tobacco Fibers

In one embodiment, the plant fibers can be obtained from a tobacco plant. Tobacco fibers may be obtained from any tobacco plant or tobacco type plant, for example, Virginia tobacco, Burley tobacco, air-cured tobacco, dark air-cured tobacco, Oriental tobacco, sun-cured tobacco, fire-cured tobacco, or mixtures thereof. Tobacco materials for use in the present disclosure may include for instance, cut leaf tobacco, a reconstituted tobacco material, or mixtures thereof, and can include tobacco hurds, stalks, and optionally leaves, as well as scraps. The tobacco fibers can be extracted fibers, dried, non-extracted fibers, or mixtures thereof.

The amount of tobacco fibers contained in the plant containing substrate can depend upon the particular application and the desired result. Tobacco fibers, such as extracted tobacco fibers, for instance, can be present in the plant containing substrate in an amount anywhere from 1% by weight to about 99% by weight, such as from about 5% by weight to about 90% by weight. For example, the tobacco fibers can be present in the plant containing substrate in an amount greater than about 10% by weight, such as in an amount greater than about 20% by weight, such as in an amount greater than about 30% by weight, such as in an amount greater than about 40% by weight, such as in an amount greater than about 50% by weight, such as in an amount greater than about 60% by weight, such as in an amount greater than about 70% by weight, such as in an amount greater than about 80% by weight, and generally in an amount less than about 95% by weight, such as in an amount less than about 90% by weight, such as in an amount less than about 85% by weight.

### Botanical Fibers

Herbal plant materials for use in the present disclosure include botanical plants, and trees, including herbs, plants and trees that may be used to form smokable fibers or herbal smokeable articles, such as cocoa tree, coffee tree or coffee bean, tea tree or tea leaf, vine, ginger, ginkgo, chamomile, tomato, ivy, mate, rooibos, cucumber, mint, a cereal such as wheat, barley or rye, or other trees such as broadleaved or resinous trees, and the like, as well as combinations thereof.

The amount of botanical fibers contained in the plant containing substrate can depend upon the particular application and the desired result. Botanical fibers, such as extracted botanical fibers, for instance, can be present in the plant containing substrate in an amount anywhere from 1% by weight to about 99% by weight, such as from about 5% by weight to about 90% by weight. For example, the botanical fibers can be present in the plant containing substrate in an amount greater than about 5% by weight, such as in an amount greater than about 10% by weight, such as in an amount greater than about 15% by weight, such as in an amount greater than about 20% by weight, such as in an amount greater than about 25% by weight, such as in an amount greater than about 30% by weight, such as in an amount greater than about 35% by weight, such as in an amount greater than about 40% by weight, and generally in an amount less than about 80% by weight, such as in an amount less than about 60% by weight, such as in an amount less than about 30% by weight.

### Cocoa Husk Fibers

In another embodiment, the plant containing substrate can contain cocoa husk fibers, such as extracted cocoa husk fibers. Extracted cocoa husk fibers, for instance, have been found to produce an aerosol very neutral in taste. Adding cocoa husk fibers to the plant containing substrate can further improve the taste of the overall product.

Cocoa materials for use in the present disclosure are obtained from Theobroma cacao, which is also referred to as the cacao tree. The cacao tree is in the evergreen family and is native to tropical regions. The cacao tree produces a fruit, referred to as a cacao pod. Cacao pods are generally yellow to orange in color and can weigh over one pound when ripe. The pod contains anywhere from 10 to about 80 cocoa beans that are used to produce chocolate, juices, jelly, and the like. After the beans are removed from the cacao pod, the cocoa beans are dried and cured or fermented by being exposed to sunlight and/or ultraviolet light. Each individual bean is covered in a husk or shell. The husk or shell is removed from the bean prior to using the bean for producing food products. The plant containing substrate of the present disclosure can contain the cocoa shells or husks, although other components of the cacao pod may also be used.

The amount of cocoa husk fibers contained in the plant containing substrate can depend upon the particular application and the desired result. Cocoa husk fibers, such as extracted cocoa husk fibers, for instance, can be present in the plant containing substrate in an amount anywhere from 1% by weight to about 90% by weight, such as from about 5% by weight to about 85% by weight. For example, the cocoa husk fibers can be present in the plant containing substrate in an amount greater than about 5% by weight, such as in an amount greater than about 10% by weight, such as in an amount greater than about 15% by weight, such as in an amount greater than about 20% by weight, such as in an amount greater than about 30% by weight, such as in an amount greater than about 40% by weight, such as in an amount greater than about 50% by weight, such as in an amount greater than about 60% by weight, and generally in an amount less than about 80% by weight, such as in an amount less than about 70% by weight, such as in an amount less than about 50% by weight.

### Bast Fibers and Wood Pulp Fibers

When treating wrapping materials in accordance with the present disclosure, the wrapper can be made from bast fibers and/or wood pulp fibers, such as softwood fibers and/or hardwood fibers.

The bast fibers can be pulped or delignified and/or bleached. Examples of bast fibers that may be used in the present disclosure include hemp fibers, flax fibers, abaca fibers, ramie fibers, cotton fibers, bamboo fibers, esparto fibers, and mixtures thereof. The bast fibers can be highly refined using, for instance, a Dutch Valley Beater.

The bast fibers can be highly refined. The bast fibers can have an average fiber length of greater than the average fiber length of the wood pulp fibers. For example, the average fiber length of the bast fibers can generally be greater than about 0.8 mm, such as greater than about 1 mm, such as greater than about 1.2 mm, such as greater than about 1.5 mm. The average fiber length of the refined bast fibers is generally less than about 4 mm, such as less than about 3.6 mm, such as less than about 3.3 mm, such as less than about 3 mm, such as less than about 2.8 mm, such as less than about 2.5 mm, such as less than about 2.3 mm, such as less than about 2.1 mm. In one particular aspect, the bast fibers can be delignified bast pulp fibers having an average fiber length of from about 1.5 mm to about 2 mm.

The wood pulp fibers, for instance, can comprise delignified fibers, such as softwood fibers, hardwood fibers, or mixtures thereof. The wood pulp fibers can have an average fiber length of generally greater than about 0.5 mm, such as greater than about 1 mm, such as greater than about 1.5 mm, such as greater than about 1.8 mm, and generally less than about 4 mm, such as less than about 3 mm, such as less than about 2.5 mm, such as less than about 2.35 mm.

### Plant Fiber Mixtures

Any of the fibers described above can be combined together to form the plant containing substrate. For example, Cannabaceae fibers can be combined with tobacco fibers and/or cocoa husk fibers. Alternatively, in one embodiment, the Cannabaceae fibers and/or tobacco fibers can be combined with botanical fibers.

Optionally, Cannabaceae fibers, tobacco fibers, botanical fibers, cocoa husk fibers or mixtures thereof can be combined with strength building fibers, such as any suitable cellulose fibers, such wood pulp fibers, regenerated cellulose fibers, other plant fibers, and the like. The strength building fibers can comprise, for instance, wood pulp fibers, bast fibers, or mixtures thereof.

The strength building fibers can be combined with, for instance, extracted plant fibers. The extracted plant fibers can be non-pulped meaning that the fibers have not been fed through a pulping process in which the fibers are combined with an alkaline material (Kraft process) or an acid material (Sulfite process) and cooked in a digester. The strength building fibers, on the other hand, can be pulped or delignified.

In one aspect, the extracted plant fibers and strength building fibers can be combined together and refined together prior to forming the web. Alternatively, each fiber can be refined separately. In still another embodiment, each fiber can be refined separately, combined together, and then refined a further amount.

In one aspect, the above plant fibers can be combined with water or an aqueous solution to form a slurry. For instance, the strength building fibers, such as delignified cellulosic fibers, can be combined with extracted plant fibers in forming the slurry. The fiber slurry is then used to form a continuous web that has the characteristics of paper. For example, in one embodiment, the fiber slurry can be fed to a papermaking process that can include a forming wire, gravity drain, suction drain, a felt press, and a dryer, such as a Yankee dryer, a candryer or the like. In one aspect, the fiber slurry is formed into a continuous sheet on a Fourdrinier table.

The fiber slurry containing the extracted fibers and the strength building fibers, for instance, can be laid onto a porous forming surface and formed into a sheet. Excess water can be removed by a gravity drain and/or a suction drain. In addition, various presses can be used to facilitate water removal. The formed sheet can be dried and further treated.

In one aspect, the fibers are all combined together in an aqueous solution and used to form a web through a wetlaid process. In this manner, a substantially homogeneous fiber distribution is achieved. The amount of each fiber contained in the resulting web can vary. In general, the web formed according to the present disclosure can contain extracted plant material in an amount greater than about 40% by weight and in an amount up to about 90% by weight, including all increments of 1% by weight therebetween. For example, the extracted plant fibers can be contained in the web in an amount greater than about 45% by weight, such as in an amount greater than about 50% by weight, such as in an amount greater than about 55% by weight, such as in an amount greater than about 60% by weight, such as in an amount greater than about 65% by weight, such as in an amount greater than about 70% by weight, such as in an amount greater than about 75% by weight, such as in an amount greater than about 80% by weight, such as in an amount greater than about 85% by weight. The extracted plant fibers can be contained in the web in an amount less than about 80% by weight, such as in an amount less than about 75% by weight, such as in an amount less than about 70% by weight.

When incorporated into the substrate, the strength building fibers can be present in the web generally in an amount up to about 50% by weight. The strength building fibers are generally present in the web in an amount greater than about 3% by weight, such as greater than about 5% by weight, such as in an amount greater than about 10% by weight, such as in an amount greater than about 15% by weight, such as in an amount greater than about 17% by weight, such as in an amount greater than about 20% by weight, such as in an amount greater than about 23% by weight, such as in an amount greater than about 25% by weight, such as in an amount greater than about 28% by weight, such as in an amount greater than about 30% by weight. The strength building fibers are generally present in an amount less than about 35% by weight, such as in an amount less than about 30% by weight, such as in an amount less than about 25% by weight, such as in an amount less than about 20% by weight, such as in an amount less than about 18% by weight, such as in an amount less than about 15% by weight. In one embodiment, the strength building fibers are refined hemp fibers. Alternatively, the strength building fibers are softwood fibers, hardwood fibers or mixtures thereof.

### Aerosol Modifying Composition

In accordance with the present disclosure, the plant containing substrate is treated with an aerosol modifying composition that comprises an additive and surfactant mixture that dramatically facilitates incorporating the additive into the substrate in a controlled manner. The surfactant incorporated into the aerosol modifying composition provides various different benefits and advantages. For instance, the surfactant may solubilize the additive or create a stable emulsion with the additive. In this manner, the additive can be easily applied to the substrate during formation of the substrate. In addition, the surfactant can be used not only to apply the additive in a uniform, controlled manner, but also enables relatively high loading into the substrate if desired.

In general, any suitable additive can be incorporated into the aerosol modifying composition that is compatible with the surfactant. Additives well suited for use in the present disclosure include cannabinoids, terpenes, essential oils, plant extracts, or mixtures thereof.

In one embodiment, an additive such as a cannabinoid can be incorporated into the plant containing substrate. A cannabinoid may include all types of cannabidiols (CBD) and all types of tetrahydocannabinols (THC). Other cannabinoids include all types of cannabigerol (CBG), cannabichromene (CBC), cannabicyclol (CBL), cannabielsoin (CBE), cannabinol (CBN), cannabinodiol (CBND), cannabitriol (CBT), cannabifuran (CBF), CBGA cannabigerolic acid, CBDA cannabidiolic acid, CBCA cannabichromenic acid, and tetrahydrocannabinolic acids. In accordance with the present disclosure, in one embodiment, THC can be applied to the plant containing substrate of the present disclosure, CBD can be applied to the plant containing substrate or, alternatively, both THC and CBD can be applied to the plant containing substrate.

In addition to THC and CBD, various other cannabinoids can also be incorporated into an aerosol delivery composition and applied to the plant containing substrate in accordance with the present disclosure. For instance, other cannabinoids contained in Cannabis include cannabichromene, cannabinol, cannabigerol, tetrahydrocannabivarin, cannabidivarin, cannabidiolic acid, other cannabidiol derivatives, and other tetrahydrocannabinol derivatives. The above cannabinoids can be used singularly or in any combination and applied to the plant containing substrate.

Each cannabinoid can be applied to the plant containing substrate in an amount greater than about 0.001% by weight and generally less than about 30% by weight. For instance, each cannabinoid can be applied in an amount greater than about 0.5% by weight, such as in an amount greater than about 1% by weight, such as in an amount greater than about 5% by weight, such as in an amount greater than about 8% by weight, such as in an amount greater than about 10% by weight, such as in an amount greater than about 12% by weight, such as in an amount greater than about 15% by weight, such as in an amount greater than about 18% by weight, such as in an amount greater than about 20% by weight, and generally in an amount less than about 28% by weight, such as less than about 25% by weight, such as less than about 22% by weight, such as less than about 20% by weight, such as less than about 18% by weight, such as less than about 15% by weight, such as less than about 12% by weight, such as less than about 10% by weight, such as less than about 8% by weight, such as less than about 5% by weight. The cannabinoid added to the aerosol modifying composition can be in the form of an oil, an emulsion, or a solid, such as a crystallized solid.

Another additive that can be added to the aerosol modifying composition and applied to the plant containing substrate are various flavorings, especially terpenes. A terpene or a blend of terpenes, for instance, can be used to develop desirable aromas and indicate to the user the quality of the product. One or more terpenes can also improve the sensory reaction to inhaling an aerosol created by the reconstituted material.

Various different terpenes can be applied to the plant containing substrate. Such terpenes include but are not limited to pinene, humulene, b-caryophyllene, isopulegol, guaiol, nerylacetate, neomenthylacetate, limonene, menthone, dihydrojasmone, terpinolene, menthol, phellandrene, terpinene, geranylacetate, ocimene, myrcene, 1,4-cineole, 3-carene, linalool, menthofuran, perillyalcohol, pinane, neomenthylaceta, alpha-bisabolol, borneol, camphene, camphor, caryophyllene oxide, alpha-cedrene, beta-eudesmol, fenchol, geraniol, isoborneol, nerol, sabinene, alpha-terpineol, and mixtures thereof.

In one embodiment, various different terpenes can be blended together in order to mimic the ratios of terpenes found in natural cannabaceae plants. For instance, from about 2 to about 12 terpenes can be blended together and applied to the plant containing substrate. Each terpene can be applied to the plant containing substrate in an amount greater than about 0.001% by weight and generally less than about 20% by weight. For instance, each terpene can be applied in an amount from about 0.01% by weight to about 15% by weight, such as in an amount of from about 0.1% by weight to about 5% by weight, such as in an amount of from about 0.1% by weight to about 2% by weight. For instance, each terpene can be applied in an amount from about 0.1% to about 1.1% by weight.

Exemplary blends of terpenes include alpha-pinene, beta-caryophyllene, and beta-pinene; alpha-humulene, alpha-pinene, beta-caryophyllene, beta-pinene, and guaiol; beta-caryophyllene, beta-pinene, and d-limonene; beta-caryophyllene, beta-pinene, and nerolidol; beta-caryophyllene, beta-pinene, d-limonene, and terpinolene; alpha-bisabolol, alpha-pinene, beta-caryophyllene, beta-myrcene, beta-pinene, and d-limonene; beta-caryophyllene, beta-pinene, and p-cymene; alpha-humulene, beta-caryophyllene, beta-pinene, d-limonene, linalool, and nerolidol; beta-caryophyllene and beta-pinene; beta-caryophyllene, beta-myrcene, and terpinolene; and alpha-pinene, beta-caryophyllene, beta-pinene, d-limonene; alpha-humulene, alpha-pinene, beta-caryophyllene, beta-myrcene, beta-pinena, d-limonene, and guaiol.

In another embodiment, various oils and extracts may be incorporated into aerosol modifying composition and applied to the plant containing substrate. Such oils can include, for instance, Cannabis oil, olive oil, sunflower oil, corn oil, essential oils, coconut oil, almond oil, black seed oil, and the like. Examples of particular additives, which may in the form of an extract and/or an oil include nicotine, sugars, licorice extracts, menthol, honey, coffee, maple syrup, tobacco extracts, botanical extracts, tea, fruit extracts, flavorings such as clove, anise, cinnamon, sandalwood, geranium, rose oil, vanilla, caramel, cocoa, lemon oil, cassia, spearmint, fennel, or ginger, fragrances or aromas such as cocoa, vanilla, and caramel, medicinal plants, vegetables, spices, roots, berries, bar, seeks, anise oil, clove oil, carvone and the like, artificial flavoring and fragrance materials such as vanillin, and mixtures thereof.

Each oil or extract can be applied to the plant containing substrate in an amount greater than about 0.001% by weight and generally less than about 20% by weight. For instance, each oil can be applied in an amount from about 0.01% by weight to about 15% by weight, such as in an amount of from about 0.1% by weight to about 8% by weight. For instance, each oil can be applied in an amount from about 0.5% to about 3% by weight.

Suitable surfactants that are incorporated into the aerosol modifying composition generally include surfactants that are approved for use with food products, smoking articles, and/or heat but not burn products. In one aspect, the surfactant can be a non-ionic surfactant, which refers to a surface active molecule comprising covalently bonded oxygen-containing hydrophilic groups and having no net electrical charge in its formulation. Amphoteric surfactants may also be used in some applications. In one aspect, the surface active molecule does not undergo any ionization when dissolved in water. Examples of surfactants may include esters of fatty acids, such as an ethoxylated sorbitan of a fatty acid, or an ethoxylate of a hydrogenated castor oil. Other non-ionic surfactants may include one or more polysorbates. For instance, the polysorbate may be a polyoxyethylene sorbitan monolaurate, a polyoxyethylene sorbitan monopalmitate, a polyoxyethylene sorbitan monostearate, a polyoxyethylene sorbitan monostearate, a polyoxyethylene sorbitan monooleate, or mixtures thereof.

In one embodiment, the surfactant is a biosurfactant derived from a plant, animal or microorganism. Biosurfactants can be produced as part of cell membranes or produced extracellularly by various organisms such as bacteria and fungi. In one aspect, the biosurfactant contains hydrophobic nonpolar moieties including unsaturated, saturated, and/or oxidized lipids or fatty acids, and hydrophilic elements, which may include amino acids, carbohydrates, phosphates or cyclic peptides. These surfactants can be generally classified according to their chemical structure into glycolipids, lipopeptides, phospholipids, fatty acids and polymeric compounds.

Examples of biosurfactants include non-ionic surfactants such as a methyl glucose sesquistearate (e.g. PEG-20 methyl glucose sesquistearate), sorbolilipids, sophorolipids, rhamnolipids, mannosyl-erythritol lipids, chlamidocins, surfactins, lysininis G, and pengine-like lipid peptides.

In still another embodiment, the biosurfactant is a lecithin. Lecithin is a lipid substance found in animal and plant tissues such as, for example, egg yolk and soybean. Lecithin is composed of various constituents including, but not limited to, phospholipids, such as, for example, phosphatidyl choline (“PC”), phosphatidyl inositol (“PI”), and phosphatidyl ethanolamine (“PE”). The amphiphilic properties of lecithin make the substance well suited for use as a dispersant in the present disclosure. Lecithins suitable for use in the disclosed compositions and methods include, but are not limited to, crude filtered lecithin, fluid lecithin, de-oiled lecithin, chemically and/or enzymatically modified lecithin, standardized lecithin, and blends of any thereof.

The process of the present disclosure offers various advantages and benefits. For instance, the process is well suited for incorporating an additive into a plant containing substrate in any particular pattern. For example, the concentration of the additive throughout the plant containing substrate can be relatively the same and exhibit concentration variations of less than about 5%, such as less than about 2%, such as less than about 1%, such as less than about 0.25% by weight. The process of the present disclosure is also very efficient. Additives can be incorporated into plant containing substrates very quickly. For example, in one embodiment, the process can take less than about 15 minutes, such as less than about 10 minutes, such as even less than about 5 minutes in order to incorporate the additive into the material.

The amount of one or more surfactants and the amount of one or more additives contained in the additive and surfactant mixture (which excludes all other components) can vary depending on many factors including the particular components being combined together, their compatibility, the amount of additive to be incorporated into the substrate, and the like. For instance, the surfactant may be present in the additive and surfactant mixture in an amount of from about 1% by weight to about 99% by weight, including all increments of 1% by weight therebetween. The surfactant, for example can be present in the additive and surfactant mixture in an amount greater than about 5% by weight, such as in an amount greater than about 10% by weight, such as in an amount greater than about 15% by weight, such as in an amount greater than about 20% by weight, such as in an amount greater than about 25% by weight, such as in an amount greater than about 30% by weight, such as in an amount greater than about 35% by weight, such as in an amount greater than about 40% by weight, such as in an amount greater than about 45% by weight, such as in an amount greater than about 50% by weight, such as in an amount greater than about 55% by weight, such as in an amount greater than about 60% by weight, such as in an amount greater than about 65% by weight, such as in an amount greater than about 70% by weight, such as in an amount greater than about 75% by weight, and generally in an amount less than about 85% by weight, such as in an amount less than about 70% by weight, such as in an amount less than about 65% by weight, such as in an amount less than about 55% by weight such as in an amount less than about 35% by weight, such as in an amount less than about 10% by weight, such as in an amount less than about 8% by weight, such as in an amount less than about 5% by weight, such as in an amount less than about 3% by weight, such as in an amount less than about 1% by weight.

The additive and surfactant mixture contains the additive in an amount of from about 0.01% by weight to about 99.5% by weight, including all increments of 1% by weight therebetween. The additive, for example can be present in the additive and surfactant mixture in an amount greater than about 0.5% by weight, such as in an amount greater than about 1% by weight, such as in an amount greater than about 2% by weight, such as in an amount greater than about 5% by weight, such as in an amount greater than about 8% by weight, such as in an amount greater than about 10% by weight, such as in an amount greater than about 15% by weight, such as in an amount greater than about 20% by weight, such as in an amount greater than about 25% by weight, such as in an amount greater than about 30% by weight, such as in an amount greater than about 35% by weight, such as in an amount greater than about 40% by weight, such as in an amount greater than about 45% by weight, such as in an amount greater than about 50% by weight, such as in an amount greater than about 55% by weight, such as in an amount greater than about 60% by weight, such as in an amount greater than about 65% by weight, such as in an amount greater than about 70% by weight, such as in an amount greater than about 75% by weight, and generally in an amount less than about 92% by weight, such as in an amount less than about 80% by weight, such as in an amount less than about 50% by weight, such as in an amount less than about 30% by weight such as in an amount less than about 20% by weight, such as in an amount less than about 10% by weight, such as in an amount less than about 8% by weight, such as in an amount less than about 5% by weight, such as in an amount less than about 3% by weight, such as in an amount less than about 1% by weight. The additive serves as an aerosol modifying agent that becomes part of the aerosol when the aerosol generating product is consumed by a user.

In addition to the surfactant and the additive, the aerosol modifying composition can also include other components. For example, the aerosol modifying composition can also contain water and can form an aqueous solution.

In one embodiment, water can be initially contained in the aerosol modifying composition to form a concentrate that is then diluted with more water when applied to the substrate. The concentrate, for instance, can contain water in an amount of greater than about 3% by weight and generally less than about 80% by weight. For instance, water can be present in the concentrate in an amount greater than about 5% by weight, such as greater than about 10% by weight, such as greater than about 15% by weight and generally less than about 70% by weight, such as less than about 50% by weight, such as less than about 30% by weight.

In one embodiment, the additive and surfactant mixture may be heated to a temperature of about 40° C. to about 90° C. in order to produce a homogeneous dispersion. The mixture can be heated prior to or after water addition.

The aerosol modifying composition comprising an additive and surfactant mixture as described above can be applied to the plant containing substrate using any suitable method or technique. For instance, the substrate can be sprayed, coated, dipped, printed, or the like with the aerosol modifying composition. Printing processes that may be used include flexographic printing, gravure printing, and the like.

In one aspect, the aerosol modifying composition is applied to the substrate using a size press as the substrate is being formed and prior to drying. The size press may comprise a size press with an applicator roll, a size press with a rod applicator, or the like. In one embodiment, the substrate can be dipped into a bath solution containing the aerosol modifying composition for application. In addition, when using a size press, the substrate can be passed through rollers that press the composition into the substrate and optionally remove excess liquid. In one embodiment, the size press may be a flooded nip size press such that an excess of aerosol modifying composition is present at the nip such that a gap may be flooded. In addition, the rolls of the size press can be controlled or regulated to obtain a desired add-on amount.

The process of the present disclosure allows for precise control over the amount of additive incorporated into the plant containing substrate due to the homogenous characteristics of the aerosol modifying composition. The additive concentration within the resulting material can also be easily adjusted and controlled according to the process with a high degree of efficacy, especially in relation to conventional methods without the use of the additive and surfactant mixture.

### Aerosol Generating Product

The cannabaceae, tobacco, cocoa shells, and botanical plants contain plant fibers which, when formed according to the present disclosure, are well suited to forming substrates and web materials. The substrate can be an aerosol producing filler or a wrapper that surrounds the filler.

### Reconstituted Material

In one embodiment, the plant fibers from at least one of the cannabaceae, tobacco, cocoa shells, and herbal plants are optionally sized or ground and then subjected to an extraction process for removing water soluble components and forming a reconstituted material. The extracted plant fibers can then be, optionally, combined with web building fibers and formed into a substrate, such as a wrapper or a reconstituted filler. The substrate can optionally be treated with the soluble extract obtained from the plant fibers. In fact, the aerosol modifying composition can be combined with the extract and applied to the substrate using, for instance, a size press.

Alternatively, the extract obtained from the plant fibers can be discarded and not recombined with the water insoluble fibers and other materials.

The reconstituted material is then dried and formed into an aerosol generating material, such as an aerosol producing filler. The aerosol generating product can then optionally be combined with various other components. For instance, the material can be treated with various aerosol delivery agents and/or combined with various other aerosol or smoking fillers, such as plant materials or other herbal fillers.

The resulting aerosol generating material made in accordance with the present disclosure can then be used in numerous different types of consumer products. For instance, in one embodiment, the aerosol generating product can be incorporated into smoking articles, such as cigarettes, cigarillos, cigars, and the like. Of particular advantage, the aerosol generating product of the present disclosure can be incorporated into devices that heat the material without burning the material to produce an aerosol that is inhaled. The aerosol generating material can be cut, shredded, or otherwise processed into a form best suited for the particular application and product.

In producing a reconstituted material for an aerosol generating product of the present disclosure, plant fibers from at least one of the cannabaceae, tobacco, cocoa shells, and herbal plants are first collected and optionally reduced in size. In one embodiment, plant fibers can be subjected to a grinding operation, milling operation or beating operation that can reduce the size of the plant fibers and/or reduce the plant into individual fibers. For example, in one embodiment, plant fiber can be fed to a hammer mill that beats the plant materials against a screen for producing a fibrous material. Reducing the size of the plant fibers, however, may not be necessary when using a cannabaceae extracted byproduct that may have already been subjected to a size reduction extraction step.

After the plant fibers are optionally reduced in size, the fibers are subjected to an extraction process for removing water soluble components. The extraction process can provide various different benefits. For instance, the extraction process can remove constituents contained in the plant material that are irritants when inhaled through an aerosol. In this manner, the extraction process can dramatically reduce from the aerosol produced by the material various harsh components.

During the extraction process, the plant fibers are contacted with a solvent in order to remove the water soluble components. In one embodiment, the solvent comprises only water. In an alternative embodiment, various solvents that are water-miscible, such as alcohols (e.g., ethanol), can be combined with water to form an aqueous solvent. The water content of the aqueous solvent can, in some instances, be greater than 50 wt. % of the solvent, and particularly greater than 90 wt. % of the solvent. Deionized water, distilled water or tap water may be employed. The amount of the solvent in the solution can vary widely but is generally added in an amount from about 50 wt. % to about 99 wt. %, in some embodiments from about 60 wt. % to about 95 wt. %, and in some embodiments, from about 75 wt. % to about 90 wt. % of the solution. However, the amount of solvent can vary with the nature of the solvent, the temperature at which the extraction is to be carried out, and the type of plant furnish.

After producing the solvent/plant fiber mixture, some or all of a soluble fraction of the furnish mixture may be separated from the mixture. The aqueous solvent/plant fibers furnish mixture can be agitated by stirring, shaking or otherwise mixing the mixture in order to increase the rate of solubilization. Typically, the process is carried out for about one-half hour to about 6 hours. Process temperatures may range from about 10° C. to about 100° C., such as from about 40° C. to about 90° C.

After the plant fibers are soaked in an extractant, the insoluble plant fiber material can be mechanically separated from the soluble plant fiber liquor or mixture located in the extract using a press. Once the soluble fraction is separated from the insoluble fraction, the soluble fraction can be discarded or further processed, such as by being concentrated. The soluble fraction can be concentrated using any known type of concentrator, such as a vacuum evaporator. In one embodiment of the present disclosure, the soluble fraction can be highly concentrated. In one embodiment, for instance, the soluble fraction can be evaporated so as to have a final dry matter content of from about 10% to about 65%, such as from about 10% to about 55%, such as from about 30% to about 55%, such as from about 45% to about 55%.

The resulting concentrated soluble fraction may be used in a separate process or can be later applied onto the reconstituted plant material optionally with the aerosol modifying composition.

The resulting water insoluble fraction is generally in an unrefined state. The insoluble fraction can comprise particles and fibers. In one embodiment, the extracted insoluble fraction can be subjected to a refining process. For instance, the extracted insoluble plant fiber material can be fed through any suitable refining device, such as a conical refiner or a disk refiner. Other refining devices that may be used include a beater, such as a Valley beater. Refining can occur while the plant materials are moist or after being combined with water. For instance, in one embodiment, refining can occur while the plant fiber material is at a consistency of less than about 10%, such as less than about 5%, such as less than about 3%.

In accordance with the present disclosure, the extracted plant fiber material may optionally be combined with web building fibers in forming a fiber substrate, such as a reconstituted plant material. For example, the extracted plant fiber can be combined with water or an aqueous solution to form a slurry, or alternatively the extracted fiber may be combined in a solution to form a slurry without the incorporation of web building fibers. In some embodiments, the web building fibers may increase the tensile strength of the sheet or wrapper of reconstituted plant material. The web building fibers, such as delignified cellulosic fibers, can be combined with the plant fiber material in forming the slurry. Regardless of whether web building fibers are used, the fiber slurry is then used to form a continuous reconstituted sheet. For example, in one embodiment, the fiber slurry is fed to a papermaking process that can include a forming wire, gravity drain, suction drain, a felt press, and a dryer, such as a Yankee dryer, a drum dryer, or the like. For example, in one embodiment, the fiber slurry is formed into a continuous sheet on a Fourdrinier table. One advantage to combining the extracted plant fiber with the cellulosic fibers is that the resulting fiber furnish can be processed on conventional papermaking equipment. However, it should be noted that, in some embodiments, the extracted plant fiber is well suited for use with papermaking equipment without the addition of web building fibers.

In one embodiment, the fiber slurry is laid onto a porous forming surface and formed into a sheet. Excess water is removed by a gravity drain and/or a suction drain. In addition, various presses can be used to facilitate water removal. The formed sheet can be dried and further treated. During formation of the sheet, as described above, the aerosol modifying composition can be applied to the sheet using, for example, a size press.

Reconstituted substrates can also be made using various other different methods. For example, in one embodiment, the extracted plant fibers and optionally, web building fibers, may be extruded into a reconstituted material. Extruding allows for the formation of rods or strands in addition to sheet materials.

While, thus far, the formation of the aerosol generating product has been described by first extracting and refining the plant materials, it should be understood that one or more types of plant fibers, may be mixed during the extraction stage, such that the plant fibers undergo extraction and refining at the same time, if desired. Alternatively, if desired, each individual plant may be extracted separately, and then mixed with another type of plant fibers during the pulping/refining process.

As described above, water soluble portion can be concentrated, optionally combined with the aerosol modifying composition and applied to the extracted fibrous substrate. The amount of water soluble extracts applied to the plant material can depend upon various factors and the anticipated end use application. In general, the water soluble extracts can be applied to the reconstituted plant material in an amount that enhances the taste of the underlying material. For instance, in one embodiment, the water soluble extracts are applied to the reconstituted material such that the reconstituted material contains water soluble extracts in an amount up to about 40% by weight, such as in amount less than 30% by weight, such as in an amount less than 20% by weight, such as in an amount less than about 10% by weight, such as in an amount less than about 5% by weight, such as in an amount less than about 1% by weight and generally in an amount greater than about 0.5% by weight.

Reconstituted plant materials made in accordance with the present disclosure have excellent mechanical characteristics and have a very desirable and aesthetic appearance. In general, the reconstituted plant material has a basis weight of greater than about 10 gsm, such as greater than about 20 gsm, such as greater than about 55 gsm. The basis weight of the reconstituted plant material is generally less than about 200 gsm, such as less than about 150 gsm, such as less than about 130 gsm.

### Cast Leaf

In one aspect, the plant containing substrate can be formed according to a cast leaf process. In a cast leaf process, the plant material is ground into powder and then blended with other materials, such as a binder, and formed into a slurry. Web building fibers can be contained within the slurry if desired. To form a web of material, the slurry is transferred to a sheet forming apparatus. The sheet forming apparatus can be a continuous belt where the slurry may be continuously spread onto the belt. The slurry is distributed on the surface to form a sheet. The sheet is then dried, such as by using heat. The sheet can be wound onto a bobbin, trimmed, slitted or otherwise manipulated for forming products. The aerosol modifying composition can be applied to the substrate during formation of the cast leaf substrate and prior to drying.

### Wetlaid Webs

The plant containing substrate can also be formed through a wet lay papermaking process without the extractions step. Wetlaid webs, for instance, can be made from wood pulp fibers. The aerosol modifying composition can be applied to the substrate during formation of the wetlaid web prior to drying.

### Additional Components

In one embodiment, the plant containing substrate can further contain a humectant. The humectant can be incorporated into the plant containing substrate for various different reasons in order to provide different benefits and advantages. For instance, in one embodiment, a humectant may be incorporated into the plant containing substrate in order to improve the processability and handling of the resulting fiber substrate. In an alternative embodiment, a humectant can be added to the plant containing substrate in greater amounts so that the material is well suited for use in applications where the material is heated but not burned in order to produce an inhalable aerosol.

Various different humectants can be incorporated into the plant containing substrate. The humectant, for instance, may comprise glycerol, propylene glycol, or mixtures thereof. Other humectants that may be used include sorbitol, triethylene glycol, lactic acid, glyceryl diacetate, glyceryl triacetate, triethyl citrate, isopropyl myristate, and mixtures thereof including mixtures with glycerol and/or propylene glycol.

As described above, the amount of humectant applied to the plant containing substrate can depend upon various factors. In one embodiment, the humectant may be present on the plant containing substrate in an amount greater than about 5% by weight, such as in an amount greater than about 10% by weight, such as in an amount greater than about 15% by weight, such as in an amount greater than about 17% by weight, and generally in an amount less than about 50% by weight, such as in an amount less than about 40% by weight, such as in an amount less than about 30% by weight, such as in an amount less than about 20% by weight. When added to the plant containing substrate in an amount from about 10 to 40% by weight, such as in an amount from about 12 to about 30% by weight, such as in an amount from about 15 to about 20% by weight, the humectant serves as an aerosol generating product that facilitates formation of an aerosol when the plant containing substrate is heated without being combusted.

The plant containing substrate of the present disclosure can also contain various other optional components. For example, in one embodiment, the plant containing substrate can optionally be treated with a burn control agent. The burn control agent can control the burn rate of the material and/or can serve as an ash conditioner for improving the coherency and/or color of the ash that is produced when the material is combusted.

The burn control agent, for instance, may comprise a salt of a carboxylic acid. For example, the burn control agent may comprise an alkali metal salt of a carboxylic acid, an alkaline earth metal salt of a carboxylic acid, or mixtures thereof. Examples of burn control agents that may be used include a salt of citric acid, malic acid, lactic acid, tartaric acid, carbonic acid, formic acid, propionic acid, glycolic acid, fumaric acid, oxalic acid, malonic acid, succinic acid, nitric acid, phosphoric acid, or mixtures thereof. Particular burn controlling agents that may be used include potassium citrate, sodium citrate, potassium succinate, sodium succinate, or mixtures thereof. When present, the burn control agent can be applied to the plant containing substrate generally in an amount greater than about 0.1% by weight, such as in an amount greater than about 0.5% by weight, such as in an amount greater than about 1% by weight and generally less than about 5% by weight, such as less than about 4% by weight, such as less than about 3% by weight, such as less than about 2% by weight.

The above burn control agents, depending upon the amount applied to the plant containing substrate, may accelerate the burn rate of the material. The plant containing substrate of the present disclosure, however, has been found to have exceptionally good burn properties without the need to add a burn control agent. In fact, in one embodiment, a burn retardant may optionally be applied to the plant containing substrate. The burn retardant, for instance, may comprise a film-forming polymer, such as an alginate, guar gum, pectin, polyvinyl alcohol, a cellulose derivative, a starch, a starch derivative, or the like. Other burn retardants include diammonium phosphate, magnesium chloride, sodium carbonate, calcium sulfate, and mixtures thereof. When present, the burn retardant can be applied to the plant containing substrate in an amount greater than about 0.1% by weight, such as in an amount greater than about 0.3% by weight, and generally in an amount less than about 3% by weight, such as in an amount less than about 2% by weight.

Optionally, the substrate can also contain filler particles. Filler particles that can be used include carbonate particles, oxide particles, and mixtures thereof. Particular filler particles include, for instance, calcium carbonate particles, magnesium oxide particles and mixtures thereof. Filler particles can be present in the web generally in an amount from about 0.5% by weight to about 30% by weight, including all increments of 0.5% therebetween. For instance, filler particles can be present in an amount greater than about 1% by weight, such as in an amount greater than about 2% by weight, such as in an amount greater than about 5% by weight, and generally in an amount less than about 25% by weight, such as in an amount less than about 20% by weight, such as in an amount less than about 15% by weight, such as in an amount less than about 12% by weight, such as in an amount less than about 8% by weight. In one aspect, filler particles can be present in an amount from about 0.5% by weight to about 5% by weight, such as from about 0.5% by weight to about 2% by weight when the web contains extracted tobacco material in an amount of about 50% by weight or greater. In another aspect, the web can be completely free of any filler particles, particularly alumina particles or other aluminum-containing filler particles.

### Filler Materials

Filler materials made in accordance with the present disclosure can have a filling power of greater than about 4 cm³/g, such as greater than about 5 cm³/g, such as greater than about 6 cm³/g, and generally less than about 10 cm³/g, such as less than about 8 cm³/g. The plant containing substrate can have excellent burn properties. For instance, the plant containing substrate can have a static burn rate of greater than about 4 mm/mm, such as greater than about 5 mm/mm, and generally less than about 8 mm/mm, such as less than about 7 mm/mm.

### Wrappers

The plant containing substrate can also take the form of a wrapper that surrounds a filler material. Wrappers can contain filler particles as described above. Wrappers can have a basis weight of from about 14 gsm to about 80 gsm and can have a permeability of from about 10 CORESTA to about 100 CORESTA.

### End Use Applications

The aerosol generating material incorporating the plant containing substrate of the present disclosure and the aerosol modifying composition can be used in all different types of aerosol generating products. In one embodiment, for instance, the aerosol generating material of the present disclosure can be formed into a smokable rod and surrounded by an outer wrapper. The smoking article, or cigarette, can include a filter located at one end of the smoking article. Alternatively, the aerosol generating material can be formed into a heat but not burn stick.

In one embodiment, the plant containing substrate, such as a reconstituted plant material, is formed on a paper forming machine and is in the form of a sheet. The sheet can then be cut into strips and fed to a rotating or agitated drum. When in the drum, the plant containing substrate can be mixed with one or more humectants and a casing. The casing can contain various different flavorants or mainstream smoke enhancing elements. For instance, the casing may contain licorice, corn syrup, and/or sugar. In addition, the casing can also include the aerosol modifying composition. From the drum, the plant containing substrate can undergo a cutting or grinding process in order to reduce the material to a desired particle size. The cut plant containing substrate is sometimes referred to as cut rag. The plant containing substrate can be packaged and shipped for use in any suitable form. In one aspect, the plant containing substrate can be fed to a cigarette making machine for forming the plant containing substrate into rod-like elements.

In addition to cigarettes, aerosol generating materials made according to the present disclosure can also include cigars and cigarillos.

The plant containing substrate of the present disclosure can also be used to produce a smokeless blend product. The smokeless product can be a dry product or can contain substantial amounts of moisture.

In addition to being incorporated into smoking articles, the aerosol generating material of the present disclosure can also be packaged and sold in various other forms to consumers. For instance, in one embodiment, the aerosol generating material can be packaged and sold as a filler material in the form of strips or shreds. The filler material can then be used in pipes, as a filler in a roll-your-own smoking article, or can be used in an aerosol generating device that heats but does not combust the material.

The present disclosure may be better understood with reference to the following examples.

### EXAMPLES

The following examples demonstrate some of the advantages and benefits of the present disclosure.

### Example 1

Various surfactant and additive mixtures were formulated in accordance with the present disclosure and applied to a plant containing substrate formed from nettles plant. The additive was cannabidiol (CBD) and was in a solid crystal form. The cannabidiol included a pentyl carbon side chain. The ethoxylated hydrogenated castor oil mentioned below included 40 moles of ethylene oxide with 1 mole of hydrogenated castor oil. The following samples were produced:
Sample A: 65% CBD/35% ethoxylated hydrogenated castor oil
Sample B: 65% CBD/35% Polyoxyethylene (20) sorbitan monolaurate (i.e. polysorbate 20)
Sample C: 65% CBD/35% Polyoxyethylene (20) sorbitan monooleate (i.e. polysorbate 80)

The concentrations of CBD were solubilized with the surfactant using heat. The CBD and surfactant mixture was heated stepwise from room temperature up to 80° C. until the CBD was completely solubilized. The soluble CBD and surfactant mixture was mixed with 250 mL water at ambient temperature to confirm that no insoluble CBD was present in the aqueous solution. The mixture was coated at a ratio of about 25% to about 55% by weight on a nettle reconstituted substrate using a size press, before final drying. Samples A through C performed with excellent results.

### Example 2

A reconstituted product was made according to the following method: a surfactant and additive mixture (CBD) was initially heated to a temperature between 55° C. to 65° C. with a CBD/polyoxyethylene (20) sorbitan monolaurate at a ratio of 65% to 35% by weight.

A reconstituted nettle material was produced. The extracted fibrous fraction was refined through disk refiners. After refining, a nettle base sheet was formed from the refined slurry. A nettle extract containing the water soluble component was concentrated in an evaporator to a solid concentration of 31%. The concentrated blend of nettle extract was combined with the surfactant and CBD mixture and coated on the fibrous substrate via manual size press. In this example, the resulting product contained CBD at 4% by weight when dried for a target of 5%, based upon the dry weight of the final product.

The above process was repeated using Polyoxyethylene (20) sorbitan monooleate (i.e. polysorbate 80).

### Example 3

A reconstituted product was made according to the following method: a surfactant and additive (CBD) mixture was initially heated to a temperature between 55° C. to 65° C. with a CBD/polyoxyethylene (20) sorbitan monolaurate ratio of 65% to 35% by weight respectively.

A reconstituted nettle material was produced. The extracted fibrous fraction was refined through disk refiners. After refining, a nettle base sheet was formed from the refined slurry. A nettle extract containing the water soluble component was concentrated in an evaporator to a solid concentration of 31%. The concentrated blend of nettle extract was combined with the surfactant and CBD mixture and coated on the fibrous sheet via manual size press. In this example, the resulting product contained CBD at 18% by weight when dried for a target of 20%, based upon the dry weight of the final product.

The above process was repeated using Polyoxyethylene (20) sorbitan monooleate (i.e. polysorbate 80).

### Example 4

A reconstituted product was made according to the following method: a surfactant and additive (CBD) mixture was initially heated to a temperature between 40° C. to 45° C. with a CBD/ethoxylated hydrogenated castor oil ratio of 65% to 35% by weight, respectively.

A reconstituted nettle material was produced. The extracted fibrous fraction was refined through disk refiners. After refining, a nettle base sheet was formed from the refined slurry. A nettle extract containing the water soluble component was concentrated in an evaporator to a solid concentration of 31%. The concentrated blend of nettle extract was combined with the surfactant and CBD mixture and coated on the fibrous sheet via manual size press.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. An aerosol generating product comprising:
a plant containing substrate impregnated with an aerosol modifying composition, the aerosol modifying composition comprising an additive and surfactant mixture, the additive comprising a cannabinoid, a terpene, an essential oil, a plant extract, or mixtures thereof, the plant containing substrate comprising cannabaceae fibers, tobacco fibers, botanical fibers, bast pulp fibers, wood pulp fibers, or mixtures thereof, the surfactant comprising a non-ionic surfactant, the surfactant comprising a polysorbate, an ethoxylated oil, or mixtures thereof, the additive being incorporated into the plant containing substrate in an amount greater than about 1% by weight.

2. An aerosol generating product as defined in claim 1, wherein the plant containing substrate comprises a reconstituted material.

3. An aerosol generating product as defined in claim 1, wherein the plant containing substrate contains cannabaceae fibers.

4. An aerosol generating product as defined in claim 1, wherein the surfactant comprises an ethoxylated sorbitan of a fatty acid or a biosurfactant.

5. An aerosol generating product as defined in claim 1, wherein the surfactant comprises a polyoxyethylene sorbitan monolaurate, a polyoxyethylene sorbitan monopalmitate, a polyoxyethylene sorbitan monostearate, a polyoxyethylene sorbitan monostearate, a polyoxyethylene sorbitan monooleate, or mixtures thereof.

6. An aerosol generating product as defined in claim 1, wherein the surfactant comprises an ethoxylate of a hydrogenated castor oil or a biosurfactant.

7. An aerosol generating product as defined in claim 1, wherein the additive comprises cannabidiol.

8. An aerosol generating product as defined in claim 1, wherein the additive comprises cannabigerol type (CBG), cannabichromene type (CBC), cannabidiol type (CBD), tetrahydrocannabinol (THC), delta-9-tetrahydrocannabinol type (Δ9-THC), delta-8-tetrahydrocannabinol type (Δ8-THC), cannabicyclol type (CBL), cannabielsoin type (CBE), cannabinol type (CBN), cannabinodiol type (CBND), cannabitriol type (CBT), cannabifuran (CBF), delta-10-tetrahydrocannabinol, cannabigerolic acid, cannabidiolic acid, cannabichromenic acid, a tetrahydrocannabinolic acid or mixtures thereof.

9. An aerosol generating product as defined in claim 1, wherein the additive comprises a terpene, the terpene comprising alpha-pinene, beta-pinene, camphene, delta-3-carene, beta-myrcene, alpha-terpinene, D-limonene, terpineol, borneol, gamma-terpinene, terpinolene, linalool, isopulegol, geraniol, beta-caryophyllene, alpha-humulene, caryophyllene oxide, nerolidol, guaiol, alpha-bisabolol or mixtures thereof.

10. An aerosol generating product as defined in claim 1, wherein the additive comprises a tetrahydrocannabinol.

11. An aerosol generating product as defined in claim 1, wherein the additive is present in the plant containing substrate in an amount greater than about 2% by weight, such as greater than about 5% by weight, such as greater than about 8% by weight, such as greater than about 10% by weight, such as greater than about 12% by weight and less than about 25% by weight, such as in an amount less than about 15% by weight.

12. An aerosol generating product as defined in claim 1, wherein the plant containing substrate comprises a filler material comprising a reconstituted material in the form of a strip, strips, shreds, or mixtures thereof.

13. A process for producing a component for an aerosol generating product, the component comprising a wrapper or an aerosol producing filler, the process comprising:
applying to a plant containing substrate an aerosol modifying composition, the aerosol modifying composition comprising water blended with an additive and a surfactant mixture, the additive comprising a cannabinoid, a terpene, an essential oil, a plant extract, or mixtures thereof, the plant containing substrate comprising cannabaceae fibers, tobacco fibers, botanical fibers, bast pulp fibers, wood pulp fibers, or mixtures thereof, the surfactant comprising a non-ionic surfactant; and
drying the plant containing substrate and wherein the additive is incorporated into the plant containing substrate in an amount greater than about 1% by weight.

14. A process as defined in claim 13, wherein the surfactant comprises a polysorbate, an ethoxylated oil, a biosurfactant, or mixtures thereof.

15. A process as defined in claim 13, wherein the surfactant comprises an ethoxylate of a hydrogenated castor oil.

16. A process as defined in claim 13, wherein the surfactant comprises an ethoxylated sorbitan of a fatty acid.

17. A process as defined in claim 13, wherein the surfactant comprises a polyoxyethylene sorbitan monolaurate, a polyoxyethylene sorbitan monopalmitate, a polyoxyethylene sorbitan monostearate, a polyoxyethylene sorbitan monostearate, a polyoxyethylene sorbitan monooleate, or mixtures thereof.

18. A process as defined in claim 13, wherein the additive and surfactant mixture is heated to a temperature of about 40° C. to about 90° C.

19. A process as defined in claim 13, wherein the additive and surfactant mixture contains the additive in an amount of from about 1 percent by weight to about 90 percent by weight, such as from about 40 percent by weight to about 70 percent by weight, such as from about 55 percent by weight to about 65 percent by weight.

20. A process as defined in claim 13, wherein the plant containing substrate is formed in a wet lay process and the aerosol modifying composition is applied to the plant containing substrate prior to drying.

21. An aerosol generating device comprising a heating device and a chamber, the chamber containing the plant containing substrate as defined in claim 1, the heating device being positioned so as to heat the plant containing substrate for producing an inhalable aerosol containing the additive without burning the plant containing substrate.
